(19) United States
(12) Patent Application Publication
(10) Pub. No.: US 2004/0082600 A1
(43) Pub. Date: Apr. 29, 2004

(54) Pharmaceutical composition

(76) Inventor: Sven Schreder, Heidelberg (DE)

Correspondence Address: Millen White Zelano & Branigan, Arlington Courthouse Plaza 1, Suite 1400, 2200 Clarendon Blvd, Arlington, VA, 22201, US

(21) Appl. No.: 10/467,788

(22) PCT Filed: Jan. 23, 2002
(86) PCT No.: PCT/EP02/00609

(30) Foreign Application Priority Data
Feb. 16, 2001 (DE) 101 07 261.9

(51) Int. Cl. A61K 31/519; A61K 35/78

(52) U.S. Cl. 514/267; 424/751

(57) ABSTRACT

The invention relates to a pharmaceutical composition having high bioavailability for oral administration of 4-[4-(3-chloro-4-methoxybenzyl-amino)benzo[4,5]thieno[2,3-d]pyrimidin-2-yl]cyclohexanecarboxylic acid and/or 4-[4-(3-chloro-4-hydroxybenzylamino)benzo[4,5]thieno[2,3-d]-pyrimidin-2-yl]cyclohexanecarboxylic acid or pharmaceutically acceptable salts thereof, to a process for the preparation of the pharmaceutical composition, and to the use thereof.

The invention relates to a pharmaceutical composition for oral administration of 4-[4-(3-chloro-4-methoxybenzylamino)benzo[4,5]thieno[2,3-d]pyrimidin-2-yl]cyclohexanecanecarboxylic acid and/or 4-[4-(3-chloro-4-hydroxy-benzylamino)benzo[4,5]thieno[2,3-d]pyrimidin-2-yl]cyclohexanecarboxylic acid or pharmaceutically acceptable salts thereof, to a process for the preparation of the pharmaceutical composition, and to the use thereof.

4-[4-(3-Chloro-4-methoxybenzylamino)benzo[4,5]thieno[2,3-d]pyrimidin-2-yl]cyclohexanecarboxylic acid and 4-[4-(3-chloro-4-hydroxybenzylamino)benzo[4,5]thieno[2,3-d]pyrimidin-2-yl]cyclohexanecarboxylic acid are inhibitors of cGMP phosphodiesterase V (PDE V inhibitors). The first-mentioned compound, pharmaceutically acceptable salts thereof and a process for the preparation thereof are described in WO 99/55708 (Example 2 or p. 9, lines 1-34). The last-mentioned compound is a phase 1 metabolite of the first-mentioned compound and is likewise pharmacologically active. Both compounds are suitable for the treatment of diseases of the cardiovascular system, in particular cardiac insufficiency, and for the treatment and/or therapy of potency disorders (erectile dysfunction).

In the last-mentioned indication in particular, it is sensible only to take the medicament as required. When taken as required, it is particularly desired that the action intended by the taking occurs as quickly as possible after administration. The prerequisite for rapid onset of action is that the active ingredient is absorbed as quickly as possible in the body and rapidly increases in concentration in the blood. The aim is therefore for the maximum active ingredient concentration occurring in the blood (Cₘₐₓ) to be reached as rapidly as possible, i.e. for the time for Cₘₐₓ to be reached (tₘₐₓ) to be as short as possible.

Active ingredients can only be absorbed by the body in dissolved form. On oral administration, the active ingredients, in order to be capable of being taken up, must therefore firstly be in dissolved form in the fluids in the gastrointestinal tract. Since only the dissolved fraction of the active ingredient is taken up in each case, active ingredients which have low solubility in gastrointestinal fluids are not absorbed completely and therefore frequently have inadequate bioavailability. Administration of the active ingredient in solution is therefore particularly suitable from the theoretical point of view. However, this possibility is frequently unsatisfactory for reasons of the lack of suitable solvents having adequate dissolving power for the active ingredient, the toxicity of solvents which are suitable from the point of view of solubility, the frequently inadequate active ingredient stability of solutions, the usually inadequate dispensing accuracy of solutions and owing to the generally poor handling ability of solutions. It is a particular problem to find a solvent which has an adequate dissolving power and at the same time is also suitable from a toxicological point of view.

The compounds mentioned at the outset and pharmaceutically usable salts thereof, such as, for example, ethanolamine salts thereof, have low solubilities in aqueous media. Thus, for example, the solubility of the ethanol-amine salt of 4-[4-(3-chloro-4-methoxybenzylamino)benzo[4,5]thieno-[2,3-d]pyrimidin-2-yl]cyclohexanecarboxylic acid is 1 mg/100 ml in synthetic gastric juice, 6 mg/100 ml in synthetic intestinal juice, 22 ng/ml in phosphate buffer (pH 1.0), 1.6 ng/ml in phosphate buffer (pH 6) and 19 ng/ml in phosphate buffer (pH 7.0). These poor solubilities have the consequence that the compound can only be taken up slowly and to an inadequate extent by the body. This is associated with poor bioavailability and a slow increase in the concentration in the blood.

One way of increasing the solubility of a medicament active ingredient which has low solubility in water and thus of improving its absorption consists in comminution thereof. The active ingredient particles are reduced in size as far as possible and employed in this form in the formulation. A reduction in the particle size into the nanometer region is known as “nanonisation”. The reduction in the particle size causes an increase in the surface area available for dissolution. Furthermore, reagents are added which modify the surface of the particles and so prevent re-aggregation. These measures cause an increase in the dissolution rate and an increase in the concentration gradient between the active Ingredient solution, for example in the stomach, and the blood. Known formulations in which the particle size of the medicament active ingredient has been “nanonised” in this way and processes for nanonisation are described in U.S. Pat. No. 5,145,684, U.S. Pat. No. 5,534,270, U.S. Pat. No. 5,585,108, U.S. Pat. No. 5,662,883, U.S. Pat. No. 5,665,331 and U.S. Pat. No. 5,718,919.

Another way of increasing the bioavailability of an active ingredient is incorporation thereof into microemulsions. Known formulations which use this technique, and processes for the preparation of microemulsions are described in U.S. Pat. No. 5,141,961, U.S. Pat. No. 5,376,688, U.S. Pat. No. 5,430,017, U.S. Pat. No. 5,688,761, U.S. Pat. No. 5,505,961, U.S. Pat. No. 5,707,648, U.S. Pat. No. 5,759,566 and U.S. Pat. No. 5,912,011.

WO 95/24893 A1 discloses a pre-emulsion formulation which forms an emulsion in vivo after administration. The composition comprises a digestible oil and an emulsifier mixture which is suitable for dispersing the oil in vivo after administration. The emulsifier mixture comprises a hydrophilic emulsifier which does not suppress lipolysis in vivo, and a lipophilic emulsifier. Suitable oils and emulsifiers mentioned are a multiplicity of different substances. All the formulations listed as examples comprise soya bean oil or coconut oil as digestible oil.

WO 97/40823 A1 discloses a pre-emulsion formulation for sexual steroids comprising triglycerides or propylene glycol esters of certain fatty acids as digestible oil, a glyceride of a C₅-C₁₀-fatty acid as lipophilic emulsifier, and POE-hydrogenated castor oil as hydrophilic emulsifier.

The invention had the object of providing a novel pharmaceutical preparation for 4-[4-(3-chloro-4-methoxybenzylamino)benzo[4,5]thieno[2,3-d]-pyrimidin-2-yl]cyclohexanecarboxylic acid or pharmaceutically acceptable salts thereof and/or 4-[4-(3-chloro-4-hydroxybenzylamino)benzo[4,5]thieno-[2,3-d]pyrimidin-2-yl]cyclohexanecarboxylic acid or one of the pharmaceutically acceptable salts, which preparation is sufficiently stable and, after oral administration, ensures a rapid increase in the concentration of the active ingredient in the body and very high bioavailability of the active ingredient.

Surprisingly, this object has been achieved by combining one or more of the said compounds with a surfactant having an HLB value of between 14 and 16.7 and a surfactant having an HLB value of between 3 and 5. The invention therefore relates to a composition which, besides 4-[4-(3-chloro-4-methoxybenzylamino)benzo[4,5]thieno[2,3-d]pyrimidin-2yl]cyclohexane-carboxylic acid and/or 4-[4-(3-chloro-4-hydroxybenzylamino)benzo[4,5]thieno[2,3-d]pyrimidin-2-yl]cyclohexanecarboxylic acid or one of the pharmaceutically acceptable salts thereof as active ingredient(s), comprises a surfactant having an HLB value of between 14 and 16.7 and a surfactant having an HLB value of between 3 and 5.

Surfactants are amphiphilic, surface-active substances which have both a hydrophilic part and a lipophilic part. The HLB value (HLB=hydrophilic-lipophilic balance) attempts to characterise the hydrophilic/lipophilic properties and enables classification of the surfactants in accordance with their intended use. The HLB value is determined empirically. It can have numerical values of between 1 and 20, a high number indicating a high hydrophilic content and a low number a high lipophilic content.

According to an embodiment of the invention, the pharmaceutical composition comprises 4-[4-(3-chloro-4-methoxybenzylamino)benzo[4,5]thieno-[2,3-d]pyrimidin-2-yl]cyclohexanecarboxylic acid and/or one of its pharmaceutically acceptable salts as active ingredient(s).

According to a further embodiment of the invention, the pharmaceutical composition comprises 4-[4-(3-chloro-4-hydroxybenzylamino)benzo[4,5]-thieno[2,3-d]pyrimidin-2-yl]cyclohexanecarboxylic acid and/or one of its pharmaceutically acceptable salts as active ingredient(s).

According to a preferred embodiment, the composition according to the invention comprises ethoxylates of castor oil or hydrogenated castor oil as the surfactant having an HLB value of between 14 and 16.7 and a mixture of mono-, di- and triglycerides of saturated fatty acids as the surfactant having an HLB value of between 3 and 5.

Ethoxylates of castor oil or hydrogenated castor oil are non-ionogenic hydrophilic emulsifiers which are prepared by reaction of ethylene oxide with castor oil or hydrogenated castor oil. The principal constituents are fatty acid glycerol polyethylene glycol esters and fatty acid polyethylene glycol esters. Polyoxyethylene (40) hydrogenated castor oil (Cremophor RH 40), polyoxyethylene (60) hydrogenated castor oil (Cremophor RH 60) and polyoxyethylene (35) castor oil (Cremophor EL), for example, are commercially available. The numbers present in these names denote the molar amount of ethylene oxide employed in the preparation per mole of castor oil or hydrogenated castor oil. The composition according to the invention particularly preferably comprises polyoxyethylene (40) hydrogenated castor oil. Cremophor is a trade name of BASF AG, 67056 Ludwigshafen.

Partial glycerides are a mixture of mono-, di- and triglycerides with saturated fatty acids having from 5 to 10 carbon atoms. Preference is given to mixtures of mono-, di- and triglycerides made from caprylic and/or caproic acid. Mixtures of mono-, di- and triglycerides with caprylic acid (trade name Imwitor 988) and mixtures of mono-, di- and triglycerides with caprylic and caproic acid (medium-chain partial glycerides; trade name Imwitor 742), for example, are commercially available. The composition according to the invention particularly preferably comprises the last-mentioned mixtures. Imwitor is a trade name of Hüls AG and can be purchased, for example, from Condea Chemie GmbH, 22297 Hamburg.

Further examples of suitable surfactants having an HLB value of between 14 and 16.7 are polyoxyethylene sorbitan fatty acid esters, such as polyoxyethylene (20) sorbitan fatty acid ester (trade name Tween 20), polyoxyethylene (40) sorbitan fatty acid ester (trade name Tween 40), polyoxyethylene (60) sorbitan fatty acid ester (trade name Tween 60), polyoxyethylene (80) sorbitan fatty acid ester (trade name Tween 80); polyethylene glycerol monooleate PEG 30/Macrogol 1000 glycerol monooleate (trade name Tagat O, Goldschmidt AG) and polyethylene glycerol monooleate PEG 20/macrogol 1000 glycerol monooleate (trade name Tagat O2). Tween is a trade name of Eurochem, 45472 Mülheim an der Ruhr, Tagat is a trade name of Goldschmidt AG, 45116 Essen.

Further examples of suitable surfactants having an HLB value of between 3 and 5 are maize oil mono-, di- and triglycerides (trade name Maisine, Gattefoss\acute{e}(Deutschland) GmbH, 79576 Weil am Rhein), glycerol mono- and dioleate (trade name Tegomuls O, Goldschmidt AG, 45116 Essen), sorbitan monostearate (trade name Span 60, Brenntag/Eurochem, 45472 Mülheim an der Ruhr), sorbitan monooleate (trade name Span 80, Brenntag/Eurochem), caprylic/caproyl macrogol 8 glyceride (trade name Labrasol, Gattefossé (Deutschland) GmbH, 79576 Weil am Rhein).

According to a particularly preferred embodiment of the invention, the composition according to the invention comprises polyoxyethylene (40) hydrogenated castor oil as the surfactant having an HLB value of between 14 and 16.7 and a mixture of mono-, di- and triglycerides of caprylic and/or caproic acid as the surfactant having an HLB value of between 3 and 5.

According to an advantageous refinement of the invention, the composition comprises a solvent or solvent mixture for the active ingredient in addition to the said active ingredients and assistants.

Preferred solvent mixtures are mixtures of two or more solvents which dissolve in one another and form a homogeneous phase. Examples of suitable solvents are propylene glycol, polyethylene glycol, glycerol, ethanol and triacetin, preferably polyethylene glycol. Polyethylene glycol having an average molecular weight of from 300 to 1500, preferably having an average molecular weight of from 300 to 600, is advantageously used. Particular preference is given to polyethylene glycol having an average molecular weight of 400.

In order to facilitate oral administration, the composition according to the invention may be present in hard or soft gelatin capsules. However, the composition can likewise be taken in the form of a liquid solution. Preference is given to soft gelatin capsules as the form of administration. The invention therefore also relates, in particular, to a soft gelatin capsule containing the composition according to the invention.

If the composition is present in capsules, in particular in soft gelatin capsules, it may be necessary for a plasticiser to be present in order to prevent hardening/embrittlement of the capsule shell. This is particularly the case if assistants which withdraw water from the capsule shell and thus result in embrittlement of the capsule shell are present. Suitable plasticisers are, for example, triacetin and glycerol. Preference is given to glycerol.

According to an advantageous embodiment of the invention, the composition comprises from 0.1 to 20% by weight of one or more of the above-mentioned active ingredients, from 5 to 60% by weight of a surfactant having an HLB value of between 14 and 16.7, from 20 to 90% by weight of a surfactant having an HLB value of between 3 and 5, from 0 to 50% by weight of solvents and from 0 to 15% by weight of plasticiser.

According to a particularly advantageous embodiment of the invention, the composition comprises, based on the total composition comprising active ingredients and assistants, from 5 to 15% by weight of one or more of the above-mentioned active ingredients and, based on the assistant composition, about 40% by weight of polyoxyethylene (40) hydrogenated castor oil, about 30% by weight of a mixture of mono-, di- and triglycerides of caprylic and/or caproic acid, about 20% by weight of polyethylene glycol having an average molecular weight of 400, and about 10% by weight of glycerol.

The composition according to the invention can be prepared by firstly dissolving the active ingredient(s) in an assistant or a mixture of a plurality of assistants and subsequently mixing the active ingredient(s) with the further assistant(s), or dissolving the active ingredient(s) directly in the mixture of all assistants. The invention therefore also relates to a process for the preparation of the composition according to the invention which is characterised in that firstly the active ingredient(s) is (are) dissolved in an assistant or a mixture of a plurality of assistants and subsequently mixed with the further assistant(s), or dissolved directly in the mixture of all assistants.

The composition according to the invention can be employed for the treatment of cardiovascular diseases, in particular cardiac insufficiency, and for the treatment of erectile dysfunction. The invention therefore also relates to the use of the composition according to the invention for the treatment of cardiovascular diseases, in particular cardiac insufficiency, and for the treatment of erectile dysfunction.

The following examples explain the invention without the latter being restricted thereto.

### EXAMPLE 1

The following compositions are illustrative embodiments of the composition according to the invention. The active ingredient (4-[4-(3-chloro-4-methoxy-benzylamino)benzo[4,5]thieno[2,3-d]pyrimidin-2-yl]cyclohexanecarboxylic acid) was in each case employed as the ethanolamine salt.

Formulation A

| | |
|---|---|
| 4-[4-(3-Chloro-4-methoxybenzylamino)- | 50 mg |
| benzo[4,5]thieno[2,3-d]pyrimidin-2-yl]- | |
| cyclohexanecarboxylic acid | |
| Polyethylene (40) hydrogenated castor oil | 180 mg |
| Medium-chain partial glycerides | 135 mg |
| Polyethylene glycol 400 | 90 mg |
| Glycerol 85% | 45 mg |
| FILLING WEIGHT: | 500 mg |
| (amount introduced into the suitable soft gelatin capsules) | |

### Preparation

The assistants and active ingredient were weighed out, dissolved in a suitable vessel with stirring and introduced into soft gelatin capsules.

### Formulation B

Further examples of the composition according to the invention are compiled in Tables 1a and b. The preparation was carried out analogously to the process described for formulation A. The amounts of active ingredients and assistants present in the compositions are in each case indicated in percent by weight.

TABLE 1a

| | Example | | | | | | |
|---|---|---|---|---|---|---|---|
| | B1 | B2 | B3 | B4 | B5 | B6 | B7 |
| Active ingredient | 5 | 15 | 10 | 15 | 15 | 15 | 15 |
| (ethanolamine salt) | | | | | | | |
| Tagat O | 29 | — | — | — | — | — | — |
| Miglyol | 38 | — | — | — | — | — | — |
| Imwitor 742 | 28 | — | — | — | — | 34 | 17 |
| Cremophor RH 40 | — | 30 | — | — | — | 26 | 43 |
| Labrasol | — | 38 | 27 | 9 | 9 | — | — |
| Propylene glycol | — | 17 | — | — | 59 | 12 | 12 |
| PEG 400 | — | — | 45 | 59 | — | — | — |
| Ethanol | — | — | 18 | 17 | 17 | 13 | 13 |
| Tween 80 | — | — | — | — | — | — | — |
| Maisine | — | — | — | — | — | — | — |

TABLE 1b

| | Example | | | | | |
|---|---|---|---|---|---|---|
| | B8 | B9 | B10 | B11 | B12 | B13 |
| Active ingredient | 10 | 10 | 10 | 10 | 10 | 10 |
| (ethanolamine salt) | | | | | | |
| Tagat O | — | — | — | — | — | — |
| Miglyol | — | — | — | — | — | — |
| Imwitor 742 | 23 | 18 | 9 | — | 54 | 36 |
| Cremophor RH 40 | 27 | 54 | 72 | 54 | 18 | — |
| Labrasol | — | — | — | — | — | — |
| Propylene glycol | — | 9 | 9 | 9 | 9 | 9 |
| PEG 400 | 27 | — | — | — | — | — |
| Ethanol | 13 | 9 | — | 9 | 9 | 9 |
| Tween 80 | — | — | — | — | — | 36 |
| Maisine | — | — | — | 18 | — | — |

### EXAMPLE 2

The following comparative formulations were prepared:

### Comparative Formulation A

Comparative formulation A is the result of a formulation development in which various formulations were prepared and tested with respect to their bioavailability in dogs. Comparative formulation A proved the most suitable of the formulations tested with respect to a fast increase in the active ingredient concentration in the body and high bioavailability.

Composition:

| | |
|---|---|
| 4-[4-(3-Chloro-4-methoxybenzylamino)- | 50 mg |
| benzo[4,5]thieno[2,3-d]pyrimidin-2-yl]- | |
| cyclohexanecarboxylic acid, ethanolamine salt | |
| Cellulose, microcrystalline: | 143 mg |
| Silicon dioxide, highly disperse: | 7 mg |
| Sodium carboxymethylstarch: | 5 mg |
| Filling weight: | 220 mg |
| (amount introduced into suitable hard gelatin capsules) | |

### Preparation

1.24 g of ethanolamine salt are dissolved in 3.75 g of ethanol at 40° C. This solution is added in a suitable manner to a mixture of 3.95 g of microcrystalline cellulose and 175 mg of highly disperse silicon dioxide which had previously been passed through a 100 mesh screen. The resultant mixture is dried at room temperature for 12 hours, mixed with 122 mg of sodium carboxymethylstarch and introduced into hard gelatin capsules.

### Comparative Formulation B

As a further comparative formulation, an active ingredient solution was selected. This is particularly advantageous with respect to a fast increase in the concentration of the active ingredient in the body since the active ingredient can be absorbed immediately without having to be dissolved in advance.

| | |
|---|---|
| 4-[4-(3-Chloro-4-methoxybenzylamino)- | 50 mg |
| benzo[4,5]thieno[2,3-d]pyrimidin-2-yl]- | |
| cyclohexanecarboxylic acid: | |
| Propane-1,2-diol | 10.345 g |
| (50 mg/10 ml) | |

The preparation was carried out by dissolving the stated amount of active ingredient in the stated amount of solvent.

### EXAMPLE 3

### Bioavailability Study

Formulation A and comparative formulations B and C were tested for their bioavailability in an open triple cross-over study on 9 healthy male test subjects aged from 18 to 35 years and having a body weight of from 60 to 100 kg. Each of the formulations was taken once by each test subject. After they had taken a formulation, blood samples were taken from each of the test subjects at suitable time intervals over a period of 72 hours, and the active ingredient concentrations in the blood samples were investigated. Based on the values obtained, the change in blood levels over time after taking the respective formulation were drawn up for each test subject. A wash-out phase of 7 days was included in each case between administration of the different formulations.

The results (Cₘₐₓ, tₘₐₓ and AUC) are shown in Table 2.

Cₘₐₓ describes the maximum concentration of the active ingredient in the blood level (blood level maximum), and tₘₐₓ describes the time interval from administration of the medicament to the occurrence of the blood level maximum (Cₘₐₓ). AUC (=area under curve) denotes the area under the blood level curve and provides information on the extent to which the amount of active ingredient present in the medicament enters the body. The AUC is thus a suitable parameter for determining the bioavailability of the active ingredient. SD denotes the standard deviation.

TABLE 2

| | | Comparative | Comparative |
|---|---|---|---|
| | Formulation A | formulation A | formulation B |
| Cₘₐₓ | 1434 ng/ml | 661 ng/ml | 1085 ng/ml |
| | SD: 39.4% | SD: 70.2% | SD: 41.2% |
| tₘₐₓ | 1.2 h | 2.4 h | 1.8 h |
| | SD: 34.7% | SD: 41.0% | SD: 47.8% |
| AUC_{(0-24 h)} | 3887 ng/ml h | 2675 ng/ml h | 3746 ng/ml h |
| | SD: 44.8% | SD: 54.3% | SD: 36.7% |

The results show significantly higher Cₘₐₓ values and significantly lower tₘₐₓ values for the composition according to the invention (formulation A) compared with the comparative formulations. The concentration of the active ingredient in the body thus increases significantly more quickly with the composition according to the invention and gives rise to significantly higher blood level maxima than with the comparative formulations. Further, the composition according to the invention also gives rise to higher AUC values and thus higher bioavailability than the comparative formulations. In particular, the significantly better results compared with comparative formulation B were unexpected, since comparative formulation B is a solution which, as such, already offers optimum prerequisites for rapid and complete absorption.

1) Pharmaceutical composition comprising 4-[4-(3-chloro-4-methoxybenzyl-amino)benzo[4,5]thieno[2,3-d]pyrimidin-2-yl]cyclohexanecarboxylic acid and/or 4-[4-(3-chloro-4-hydroxybenzylamino)benzo[4,5]thieno[2,3-d]-pyrimidin-2-yl]cyclohexanecarboxylic acid or one of the pharmaceutically acceptable salts thereof as active ingredient(s), a surfactant having an HLB value of between 14 and 16.7 and a surfactant having an HLB value of between 3 and 5.

2) Pharmaceutical composition according to claim 1, characterised in that 4-[4-(3-chloro-4-methoxybenzylamino)benzo[4,5]thieno[2,3-d]pyrimidin-2-yl]cyclohexanecarboxylic acid and/or one of its pharmaceutically acceptable salts is present as active ingredient(s).

3) Pharmaceutical composition according to claim 1, characterised in that 4-[4-(3-chloro-4-hydroxybenzylamino)benzo[4,5]thieno[2,3-d]pyrimidin-2-yl]cyclohexanecarboxylic acid and/or one of its pharmaceutically acceptable salts is present as active ingredient(s).

4) Pharmaceutical composition according to claims 1 to 3, characterised in that ethoxylates of castor oil or hydrogenated castor oil are present as the surfactant having an HLB value of between 14 and 16.7, and a mixture of mono-, di- and triglycerides of saturated fatty acids is present as the surfactant having an HLB value of between 3 and 5.

5) Pharmaceutical composition according to claim 4, characterised in that polyoxyethylene (40) hydrogenated castor oil is present as the surfactant having an HLB value of between 14 and 16.7, and a mixture of mono-, di- and triglycerides of caprylic and/or caproic acid is present as the surfactant having an HLB value of between 3 and 5.

6) Pharmaceutical composition according to claims 1 to 5, characterised in that a solvent or solvent mixture is additionally present.

7) Pharmaceutical composition according to claim 6, characterised in that polyethylene glycol is present as the solvent.

8) Pharmaceutical composition according to claim 7, characterised in that polyethylene glycol having an average molecular weight of from 300 to 600, preferably 400, is present as the solvent.

9) Pharmaceutical composition according to claims 1 to 8, characterised in that a plasticiser is additionally present.

10) Pharmaceutical composition according to claim 9, characterised in that glycerol is present as the plasticiser.

11) Pharmaceutical composition according to claims 1 to 10, characterised in that it comprises from 0.1 to 20% by weight of one or more of the above-mentioned active ingredients, from 5 to 60% by weight of a surfactant having an HLB value of between 14 and 16.7, from 20 to 90% by weight of a surfactant having an HLB value of between 3 and 5, from 0 to 50% by weight of solvents and from 0 to 15% by weight of plasticiser.

12) Pharmaceutical composition according to claims 1 to 11, characterised in that it comprises, based on the total composition comprising active ingredients and assistants, from 5 to 15% by weight of one or more of the above-mentioned active ingredients and, based on the assistant composition, about 40% by weight of polyoxyethylene (40) hydrogenated castor oil, about 30% by weight of a mixture of mono-, di- and triglycerides of caprylic and/or caproic acid, about 20% by weight of polyethylene glycol having an average molecular weight of 400, and about 10% by weight of glycerol.

13) Process for the preparation of a composition according to claims 1 to 12, characterised in that firstly the active ingredient(s) is (are) dissolved in an assistant or a mixture of a plurality of assistants and subsequently mixed with the further assistant(s), or dissolved directly in the mixture of all assistants.

14) Capsule, characterised in that it contains the pharmaceutical composition according to claims 1 to 12.

15) Use of the composition according to claims 1 to 12 and the capsule according to claim 14 for the treatment of cardiovascular diseases, in particular cardiac insufficiency, and for the treatment of erectile dysfunction.
